(19) United States
(12) Patent Application Publication
(10) Pub. No.: US 2025/0325831 A1
(43) Pub. Date: Oct. 23, 2025

(54) THERAPEUTIC ENVIRONMENT SENSING AND/OR ALTERING DIGITAL HEALTH PLATFORM

(71) Applicant: Circadian OS, Inc., New York, NY (US)

(72) Inventors: Sofia AXELROD, Brooklyn, NY (US); Grant Kenji LARSEN, Millbrook, NY (US)

(21) Appl. No.: 18/965,364

(22) Filed: Dec. 2, 2024

Related U.S. Application Data
(63) Continuation of application No. 18/621,696, filed on Mar. 29, 2024, now abandoned.
Application No. 18/621,696 is a continuation of application No. PCT/US22/45385, filed on Sep. 30, 2022.
(60) Provisional application No. 63/250,822, filed on Sep. 30, 2021.

(51) Int. Cl. A61N 5/06; A61M 21/00; A61M 21/02; G16H 10/60; G16H 20/40; G16H 40/63

(52) U.S. Cl. CPC A61N 5/0618; A61M 21/02; G16H 10/60; G16H 20/40; G16H 40/63; A61M 2021/0044; A61M 2205/3327; A61M 2205/3375; A61M 2205/50; A61M 2230/005; A61N 2005/0626; A61N 2005/0663; A61N 2005/0667

(57) ABSTRACT

A therapeutic lighting, sensing, and software system may aid users in various ways. The system may include a lamp in signal communication with a backend computing device. The system may include a secondary computing device in signal communication with the backend computing device. The backend computing device may be used to help control the lamp, based at least in part on data received from the lamp and/or the secondary computing device.

### CROSS-REFERENCE

This application is a continuation application of U.S. patent application Ser. No. 18/621,696, filed Mar. 29, 2024, which is a continuation application of PCT Application No. PCT/US2022/045385, Publication No. WO 2023056016 A1, filed Sep. 30, 2022, which claims priority from U.S. Provisional Application No. 63/250,822 filed on Sep. 30, 2021, each of which is incorporated herein by reference in its entirety.

### BACKGROUND

Between 10% and 30% of the US population experiences chronic insomnia, and over 69% of workers experience workplace fatigue. Insomnia leads to increased health risks including diabetes, obesity, depression, car accidents, and early death. Prescription sleep aids often have unsatisfactory results, and more comprehensive interventions are complex, difficult to implement and inaccessible for the general population. In recent years, the impact of circadian rhythms—a body's inner clock—on sleep as well as many other health parameters has gotten increased attention.

Many physiological parameters including body temperature, blood pressure, liver function, muscle strength, mood, alertness and many hormones, including the sleep hormone melatonin, exhibit daily oscillations with a periodicity of about a day (Latin: ‘circa’=about, ‘diem’=a day). Circadian rhythms are “entrained” by so-called zeitgebers to a particular phase to promote alignment of the inner clock with the outside world. The main zeitgeber is ˜480 nm blue light. Exposure to this wavelength, which is present in daylight as well as most electrical lighting, triggers activation of the light receptor melanopsin in the ipRGCs, a special non vision-forming cell type in the retina. The light signal is transmitted from the eyes to the suprachiasmatic nucleus, a dedicated brain area which regulates most circadian processes in the body and is therefore considered the body's “master clock”.

Light resets the circadian clock, suppresses the sleep hormone melatonin, and is therefore a powerful modulator of our sleep/wake cycles. After sunset, melatonin can rise and sleep is promoted. Research shows that electrical lighting in our homes and light emitted from screens including e-readers and smartphones is highly effective in disrupting circadian rhythms, suppressing melatonin production in the evening and causing sleep loss in both adults and children, creating a link between the high prevalence of insomnia and electrical lighting. On the other hand, indoor lighting is typically not strong enough to elicit the positive physiological effects of light during the day.

Given light's therapeutic properties, including impact on circadian rhythms, light interventions have been studied as a tool to improve sleep and increase human health and well-being. Bright light therapy for insomnia as well as other health conditions including depression has been proven effective in clinical trials, and the effect of circadian lighting-increasing the aspect of 480 nm-enriched (melanopic) light during the day and decreasing melanopic light exposure in the evening and during the night—has been shown to help office and shift workers, travelers, students and adolescents, NICU babies, nursing home residents, Alzheimer patients, cancer patients and new mothers to improve sleep, reduce inflammation, improve alertness, memory, cognition and mood, reduce jetlag, feel better and be more productive.

While circadian lighting has a number of health benefits, it is not readily available for the general public. Given insomnia's pandemic proportions and the complexity of efficient interventions, new solutions are necessary to allow humans to get the rest they need in a 24/7 society with continuous electrical lighting, which disrupts sleep at night yet is not strong enough to promote positive health effects during the day.

### SUMMARY

A therapeutic lighting, sensing, and software platform may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

A therapeutic lighting, sensing and software platform may aid users in various ways. The platform may include a lamp in signal communication with a backend computing device. The platform may include a secondary computing device in signal communication with the backend computing device. The backend computing device may be used to help control the lamp, based at least in part on data received from the lamp and/or the secondary computing device.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a block diagram of an operating environment consistent with the present disclosure;

FIG. 2A illustrates a block diagram of a first edge device consistent with the present disclosure;

FIG. 2B illustrates a block diagram of a second edge device consistent with the present disclosure;

FIG. 3 is a flow chart of a method for providing therapeutic lighting, sensing, and computing systems; and

FIG. 4 is a block diagram of a system including a computing device for performing the method of FIG. 3.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of a therapeutic lighting, sensing, and digital health system, embodiments of the present disclosure are not limited to use only in this context.

### I. Platform Overview

Consistent with embodiments of the present disclosure, a therapeutic lighting, sensing, and software platform may be provided. This overview is provided to introduce a selection of concepts in a simplified form that are further described below. This overview is not intended to identify key features or essential features of the claimed subject matter. Nor is this overview intended to be used to limit the claimed subject matter's scope. The therapeutic lighting, sensing, and software platform may be used by individuals or companies to improve sleep, reduce fatigue and improve mood and other health parameters in a reactive and adaptive fashion while providing ambient illumination in an enclosed area, such as a bedroom. In particular, the therapeutic lighting, sensing, and software platform may facilitate boosting of effectiveness of certain medications, may help to mitigate at least some effects of Alzheimer's disease, and may help reduce at least some symptoms of migraine headaches.

The therapeutic lighting, sensing, and software platform may include a backend computing device. The backend computing device may have one or more data connections for receiving data. The backend computing device may store received data in a database or similar system, and may analyze the received data to provide alerts that help a user sleep better, reduce fatigue and increase mood and other health parameters in a reactive and adaptive fashion.

The therapeutic lighting, sensing, and software platform may include an edge device having one or more sensors for gathering information about conditions in the area surrounding the edge device. In some embodiments, the edge device may take the form of a lamp or other illumination device. The edge device may communicate with the backend computing device. For example, the edge device may provide data gathered from the one or more sensors to the backend computing device, and may receive one or more commands from the backend computing device to control the edge device.

The therapeutic lighting, sensing, and software platform may include a secondary computing device in signal communication with the backend computing device. In embodiments, the secondary computing device may include application software allowing a user to send and receive information to the backend computing device. In embodiments, the secondary computing device may be, for example, a smartphone, a tablet computer, a laptop computer, a personal computer, or the like. The secondary computing device may receive data from the backend computing device, and may provide one or more alerts or instructions to the user via an input/output interface, such as a speaker or display.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Therapeutic Lighting Software Platform

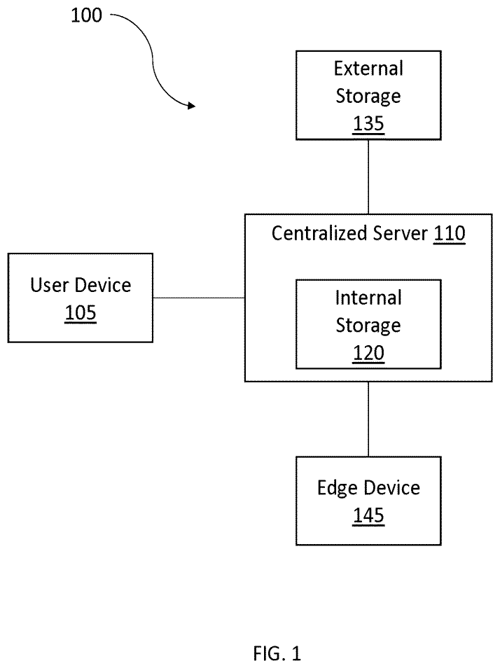

FIG. 1 illustrates one possible operating environment through which a digital health platform consistent with embodiments of the present disclosure may be provided. By way of non-limiting example, a therapeutic lighting, sensing, and software platform 100 may be hosted on a centralized server 110, such as, for example, a cloud computing service. A user 105 may access platform 100 through a software application. The software application may be embodied as, for example, but not be limited to, a website, a web application, a desktop application, and a mobile application compatible with a computing device 400.

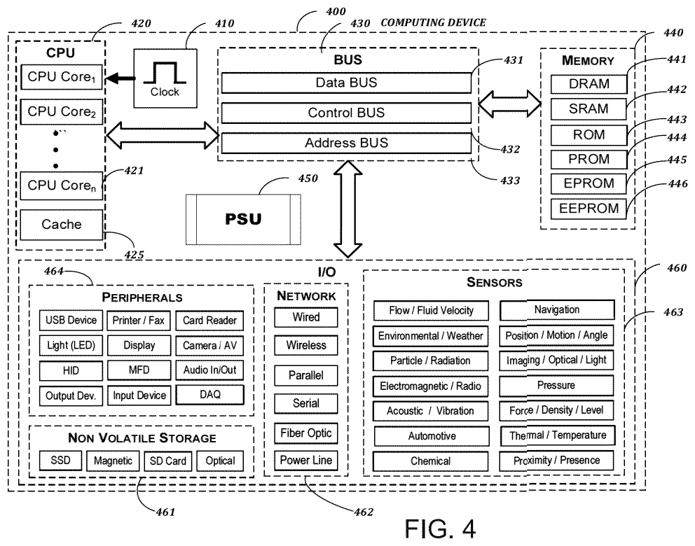

As will be detailed with reference to FIG. 4 below, the computing device through which the digital health platform may be accessed may comprise, but not be limited to, for example, a desktop computer, laptop, a tablet, or mobile telecommunications device. Though the present disclosure is written with reference to a mobile telecommunications device, it should be understood that any computing device may be employed to provide the various embodiments disclosed herein.

In embodiments, the therapeutic lighting, sensing, and software platform 100 may include an edge device 145. The edge device 145 may include one or more sensors. In embodiments, the edge device 145 may transmit data to and/or receive data from the centralized server 110.

In embodiments, the centralized server 110 may receive data from the user 105 and/or the edge device 145. The centralized server may store and/or process the received data. In some embodiments, processing the received data may include programmatic and/or algorithmic processing. Alternatively or additionally, processing the received data may include machine learning processing.

In an embodiment, the machine learning processing may include use of a machine learning engine. Machine learning includes various techniques in the field of artificial intelligence that deal with computer-implemented, user-independent processes for solving problems that have variable inputs.

In some embodiments, the machine learning engine trains a machine learning model to perform one or more operations. Training a machine learning model uses training data to generate a function that, given one or more inputs to the machine learning model, computes a corresponding output. The output may correspond to a prediction based on prior machine learning. In an embodiment, the output includes a label, classification, and/or categorization assigned to the provided input(s). The machine learning model corresponds to a learned model for performing the desired operation(s) (e.g., labeling, classifying, and/or categorizing inputs). For example, the machine learning model may be used in determining a likelihood of a transaction to complete a stage in particular amount of time.

In an embodiment, the machine learning engine may use supervised learning, semi-supervised learning, unsupervised learning, reinforcement learning, another training method, and/or combinations thereof. In supervised learning, labeled training data includes input/output pairs in which each input is labeled with a desired output (e.g., a label, classification, and/or categorization), also referred to as a supervisory signal. In semi-supervised learning, some inputs are associated with supervisory signals and other inputs are not associated with supervisory signals. In unsupervised learning, the training data does not include supervisory signals. Reinforcement learning uses a feedback system in which the machine learning engine receives positive and/or negative reinforcement in the process of attempting to solve a particular problem (e.g., to optimize performance in a particular scenario, according to one or more predefined performance criteria). In an embodiment, the machine learning engine initially uses supervised learning to train the machine learning model and then uses unsupervised learning to update the machine learning model on an ongoing basis.

In an embodiment, a machine learning engine may use many different techniques to label, classify, and/or categorize inputs. A machine learning engine may transform inputs (e.g., the augmented sensor data) into feature vectors that describe one or more properties (“features”) of the inputs. The machine learning engine may label, classify, and/or categorize the inputs based on the feature vectors. Alternatively or additionally, a machine learning engine may use clustering (also referred to as cluster analysis) to identify commonalities in the inputs. The machine learning engine may group (i.e., cluster) the inputs based on those commonalities. The machine learning engine may use hierarchical clustering, k-means clustering, and/or another clustering method or combination thereof. In an embodiment, a machine learning engine includes an artificial neural network. An artificial neural network includes multiple nodes (also referred to as artificial neurons) and edges between nodes. Edges may be associated with corresponding weights that represent the strengths of connections between nodes, which the machine learning engine adjusts as machine learning proceeds. Alternatively or additionally, a machine learning engine may include a support vector machine. A support vector machine represents inputs as vectors. The machine learning engine may label, classify, and/or categorizes inputs based on the vectors. Alternatively or additionally, the machine learning engine may use a naïve Bayes classifier to label, classify, and/or categorize inputs. Alternatively or additionally, given a particular input, a machine learning model may apply a decision tree to predict an output for the given input. Alternatively or additionally, a machine learning engine may apply fuzzy logic in situations where labeling, classifying, and/or categorizing an input among a fixed set of mutually exclusive options is impossible or impractical. The aforementioned machine learning model and techniques are discussed for exemplary purposes only and should not be construed as limiting one or more embodiments.

In an embodiment, as a machine learning engine applies different inputs to a machine learning model, the corresponding outputs are not always accurate. As an example, the machine learning engine may use supervised learning to train a machine learning model. After training the machine learning model, if a subsequent input is identical to an input that was included in labeled training data and the output is identical to the supervisory signal in the training data, then output is certain to be accurate. If an input is different from inputs that were included in labeled training data, then the machine learning engine may generate a corresponding output that is inaccurate or of uncertain accuracy. In addition to producing a particular output for a given input, the machine learning engine may be configured to produce an indicator representing a confidence (or lack thereof) in the accuracy of the output. A confidence indicator may include a numeric score, a Boolean value, and/or any other kind of indicator that corresponds to a confidence (or lack thereof) in the accuracy of the output.

In some embodiments, the centralized server 110 may receive user data from the user 105. For example, the user data may be received via the software application. In embodiments, the user data may include parameters related to the user biographic and demographic data, current user sleep habits, and/or desired user sleep habits. As a particular example, the following Table 1 shows a non-limiting selection of parameter data that may be received from the user.

TABLE 1

| | |
|---|---|
| Biographic | Age |
| | Gender |
| Sleep | Desired wake time |
| | Desired bedtime |
| | How many hours of sleep do you need per night? |
| Chronotype | Daytime preference (owl/lark) |
| | What times of day are you typically tired? |
| Light | Do you prefer well-lit or dimly lit rooms? |
| sensitivity | Do you absolutely need sunglasses on a sunny day? |
| Mood | At what times of the day are you typically in the best/ |
| | worst mood? |
| | Do you suffer from depression? |
| | Do you get depressed in the winter (seasonal affective |
| | disorder)? Is your depression worse in the winter? |
| Family | Do you live alone or with other family members? |
| Phase | Do you get up/go to bed at different times on some days |
| shifts & | (e.g., the weekend)? |
| travel | Are you a shift worker? |
| | Do you travel frequently across time zones? How often? Do |
| | you experience jetlag? |
| For shift | Enter schedules for your upcoming shiftwork days: required |
| workers: | work hours, desired wake and bed times |
| | Do you work with remote teams or teammates in other time |
| | zones? |
| For | Enter flight number and dates. How early do you have to |
| travelers: | get up at your destination? How late do you have to stay |
| | up? |
| Family | Do you need help aligning your schedule with other family |
| | members (e.g., children)? If yes, enter their info, work/ |
| | school start etc. |

The user data may also identify one or more goals. Goals the user may identify include, for example, but not be limited to, the following:
improve sleep;
improve productivity;
reduce fatigue;
combat afternoon/morning/evening slump;
change my schedule (get up earlier/later);
improve mood;
alleviate shift work fatigue;
alleviate jetlag;
align family schedules;
align remote work schedules;
align children's schedules with mine;
enhance pharmaceutical effectiveness; and
medical therapies (SAD, Alzheimer's, migraines).

The centralized server 110 may store the received user data (e.g., in an internal data store 120 and/or in an external data store 135). Alternatively or additionally, the centralized server 110 may process the received user data. The processing may include a programmatic and/or algorithmic processing and/or a machine learning processing. In particular, the centralized server 110 may generate a schedule and/or a light algorithm for the user, based at least in part on the received user data. The centralized server 110 may cause the schedule and/or the light algorithm to be displayed to the user via the software application.

In some embodiments, the centralized server 110 may receive data (e.g., sensor data) from the edge device 145. The centralized server 110 may store the received sensor data (e.g., in an internal data store 120 and/or in an external data store 135). Alternatively or additionally, the centralized server 110 may process received data. The processing may include a programmatic and/or algorithmic processing and/or a machine learning processing.

In embodiments, processing the received data from the edge device 145 may include the centralized server 110 transmitting a response to the edge device. The response may include one or more instructions for causing the edge device to perform an action. As a particular example, the edge device 145 may include a lamp, and the response may include an instruction to adjust an intensity (e.g., brightness) and/or color of the light emitted by the lamp.

In some embodiments, the centralized server 110 may control an edge device 145 associated with a particular user 105 based on a generated light algorithm (a light diet recipe) for the particular user.

### Example 2: Edge Devices

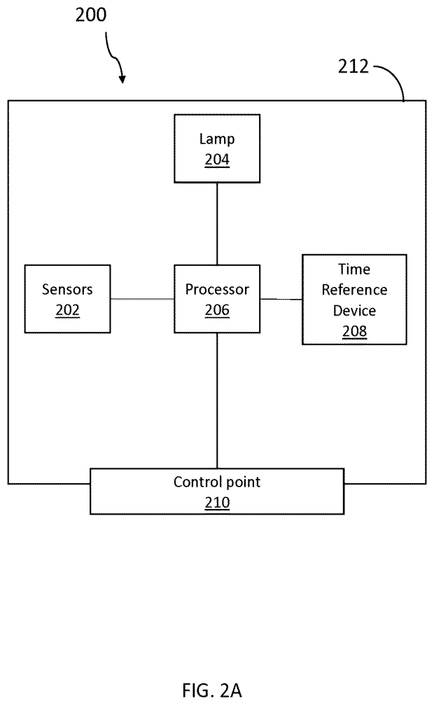

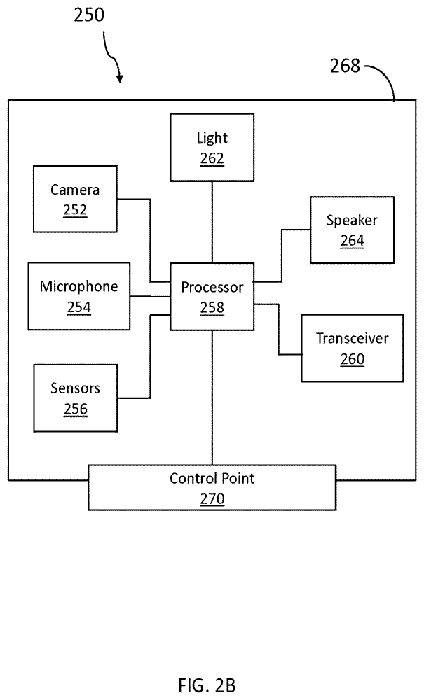

FIG. 2A is a schematic depiction of a first edge device 200 (e.g., the edge device 145). The first edge device 200 may include one or more sensors 202, a lamp 204, a processor 206, a time reference device 208, and a control point 210, arranged within a housing 212. The first edge device 200 may be an autonomous edge device, operating without direct input from a server (e.g., the centralized server 110).

In embodiments, the first edge device 200 may include one or more sensors 202. The one or more sensors may measure characteristics of ambient environment surrounding the lamp.

In some embodiments, the one or more sensors 202 may include a sensor for measuring the light intensity in the room. The sensor for measuring the light intensity may include one or more photosensors such as (but not limited to), for example, one or more photodiodes. Alternatively or additionally, the sensor for measuring the light intensity may include a more complex sensor, such as a camera. In some embodiments, the one or more sensors 202 may include an audio sensor, such as a sound level meter (e.g., an audiometer) and/or a microphone. The audio sensor may measure sound levels to determine an intensity of sound present in the environment. The one or more sensors 202 may include additional sensors and/or different sensors, without departing from the scope of the invention.

The first edge device 200 may include a lamp 204 for illuminating an area surrounding the edge device. The lamp 204 may comprise one or more light emitting diodes (LEDs). The lamp 204 may emit light at least having wavelengths in the range of 620-650 nm or more. Light having wavelengths of 620 nm-650 nm or more does not promote wakefulness during normal sleep time, but may provide sufficient illumination for a user to see the area surrounding the first edge device 200. In some embodiments, the lamp 204 may produce light having a broad spectrum of wavelengths (e.g., white light).

The first edge device 200 may include a processor 206 that receives, as input, sensor measurements from the one or more sensors 202. The processor 206 may be connected to the one or more sensors 202 so as to receive data (e.g., sensor output) from each of the one or more sensors. The processor 206 may be capable of analyzing the data received from the one or more sensors 202. In some embodiments, the analysis may optionally include using machine learning to analyze the data. That is, the inputs may be provided to a trained machine learning model capable of categorizing the received data. The machine learning model may be stored locally (e.g., at the first edge device 200) and/or remotely (e.g., at the centralized server 110). Alternatively or additionally, the analysis may include a threshold analysis and/or an algorithmic analysis instead of or in addition to the machine learning. As an example, the processor 206 may analyze data from an audio sensor (e.g., a microphone) to determine whether sounds in the room are indicative of sleep (e.g., snoring) or wakeful activities (e.g., talking, crying etc.). As another example, the processor 206 may analyze data from a camera among the sensors 202 to determine if there is movement in the vicinity of the first edge device 200. The processor 206 may produce, as output, a signal for controlling the lamp 204 based on the processed input sensor signals.

The first edge device 200 may include a time reference device 208. In some embodiments, the time reference device 208 may include a real time clock (RTC). The time reference device 208 allows the first edge device 200 to determine a current time of day, a day of the week, and/or a current season. Determining time, day, and/or season may be important for maintaining circadian rhythms and improving sleep health. In other embodiments (e.g., where no RTC is present), the time reference device may be a remote time source, or may correspond to a user input setting the time and date, wherein the processor 206 may serve as the time reference device 208 by counting clock cycles. In embodiments, the time reference device 208 may serve as an input to the processor 206 for use in providing the output signal to the lamp 204.

The first edge device 200 may include a control point 210. In some embodiments the control point may be, for example, a switch, dial, or mechanical button that a user may actuate. In other embodiments, the control point 210 may include one or more capacitive touch plates. As a particular example, the control point 210 may include a capacitive touch plate formed from a natural wood material embedded with a fine meshed copper fabric to create a conductive surface. The control point 210 may allow for touch control of the first edge device 200. The control point 210 may be used to adjust light levels emitted by the lamp 210 and/or to turn the lamp on or off. The control point 220 preferably operates silently, and may be formed from materials that damp or otherwise reduce vibrations and noise.

The edge device 200 may be substantially enclosed within a housing 212. The housing 212 may be formed from any opaque or semi-opaque, durable material. In some embodiments, the housing 212 is formed from wood. In some embodiments, a first portion of the housing 212 may be formed from a wood veneer of a particular wood species. The first portion may act as a diffuser, modulating the frequency of light emitted by the lamp 204 to reduce the blue content (e.g., light having a wavelength at or near 480-490 nm). The first portion may allow light having a wavelength near the range of 620-650 nm or higher to pass through the housing, while restricting or blocking light outside that range (e.g., light having a wavelength below 620 nm) from passing through. In some embodiments, the control point 210 may protrude at least partially from the housing 212. The housing 212 may optionally be separated from the control point 210 to define a gap sized to emit light therefrom. The gap may help to indicate, to a user, a position of the touch target (e.g., the control point 210). Alternatively, the control point 210 may directly abut the housing 212.

FIG. 2B is a schematic depiction of a second edge device 250 (e.g., the edge device 145). The second edge device 250 may include a camera 252, a microphone 254, one or more sensors 256, a processor 258, a time reference device 260, a transceiver 262, a lamp 264, and/or a speaker 266. In embodiments, the second edge device 250 is substantially enclosed within a housing 268. In embodiments the second edge device 250 is an advanced edge device that may be configured to send data to and/or receive data from a centralized server (e.g., the centralized server 110).

The second edge device 250 may include a camera 252. The camera 252 may measure an intensity of ambient light in an area surrounding the second edge device 250. Measuring intensity of ambient light may include measuring intensity of ambient light in one or more wavelength ranges. For example, the camera 252 may measure light intensity specifically in the blue range (e.g., light having a wavelength at or near 480-490 nm). In some embodiments, the camera 252 may detect motion in the area surrounding the second edge device 250 in addition to or instead of detecting the intensity of the ambient light.

In embodiments, the second edge device 250 may include a microphone 254. The microphone 254 may be used to measure sound intensity in the environment surrounding the second edge device 250. The microphone 254 may be, for example, a piezoelectric microphone, a condenser microphone, or any other transducer capable of converting sound waves to an electrical impulse.

In some embodiments, the second edge device 250 may include one or more sensors 256 instead of or in addition to the camera 252 and/or the microphone 254. In embodiments, the one or more sensors 256 may collect ambient environmental data that may affect health of the user. For example, the ambient environmental conditions measured by the one or more sensors 256 may include any conditions that may affect circadian health of a user (e.g., restlessness, trouble sleeping, etc.), and/or may affect general health of the user (e.g., Alzheimer's disease symptoms, migraines, and other health issues). As particular examples, the one or more sensors 256 may include a temperature sensor, an air pressure sensor, an audio sensor, and/or a lux meter(e.g., one or more photodiodes). Various sensors may be used to measure ambient environmental data that may affect user health.

The second edge device 250 may include a processor 258 connected to the camera 252, the microphone 254, and/or the one or more sensors 256. The processor 258 may be capable of analyzing data received from the camera 252, the microphone 254, and/or the one or more sensors 256. In some embodiments, the analysis may optionally include using machine learning to analyze the data. That is, the inputs (e.g., at least a portion of the received data) may be provided to a machine learning model capable of categorizing the received data. The machine learning model may be stored locally, at the second edge device 250, and/or at a server (e.g., the centralized server 110) in communication with the second edge device. Alternatively or additionally, the analysis may include a threshold analysis and/or an algorithmic analysis instead of or in addition to the machine learning. The processor 258 may produce, as output, a signal for controlling the lamp 264, the speaker 266, and/or any other component of the second edge device 250 based on the processed input sensor signals.

For example, the processor 258 may analyze the data received from the camera 252 to map a color space of an environment surrounding the second edge device 250 using HDR techniques. This color mapping may be based on wall color, ambient lighting, and/or light output by the second edge device 250. Specifically, the data from the camera 252 may be analyzed to measure an intensity and/or a frequency of the light in the environment.

The second edge device 250 may include a time reference device 260. In some embodiments, the time reference device 260 may include a real time clock (RTC). The time reference device 260 allows the second edge device 250 to determine a current time of day and the current season. Determining both time of day and season may be important for circadian rhythms and improving sleep health. In other embodiments (e.g., where no RTC is present), the time reference device 260 may be a remote time source, or may correspond to a user input setting the time and date, wherein the processor 258 may serve as the time reference device 260 by counting clock cycles. In embodiments, the time reference device 260 may serve as an input to the processor 258 for use in calculating the output signal.

The second edge device 250 may include a transceiver 262 for communication with other devices. The transceiver 262 may be configured to send and receives wireless signals, such as signals compliant with the International Electrical and Electronics Engineers (IEEE) 802.11 standards and/or any other wireless data transmission standards. In embodiments, the transceiver 262 may facilitate transmission of data (e.g., data received from the camera 252, the microphone 254, and/or the one or more sensors 256) to a server (e.g., the centralized server 110) or any other device connected (directly or indirectly) to the second edge device 250. In some embodiments, the transceiver 262 may be in communication with an external time source as part of the time reference device 260.

The second edge device 250 may include a lamp 264 for illuminating an area surrounding the second edge device. The lamp 264 may comprise one or more light emitting diodes (LEDs). The lamp 264 may emit light at least having wavelengths in the range of 620-650 nm or higher. Light having these wavelengths does not promote wakefulness during normal sleep time, but provides sufficient illumination to see the area surrounding the second edge device 250. In some embodiments, the lamp 264 may emit light having a broad spectrum of wavelengths (e.g., white light).

In some embodiments, the lamp 264 may include an array of RGBW LEDs that are individually controllable and addressable. The lamp 264 may further include an array of micromirrors to create a light pattern to improve effects on the human circadian and melanopic system.

In some embodiments, the lamp 264 may project light onto a wall or ceiling in the manner of a torchiere. This may maximize an area of the lighting surface has, to keep the light density low, even at high levels of illumination, to further the circadian goals while helping to minimize glare.

In some embodiments, the lamp 264 may include an array of LEDs that are individually controllable and addressable. In embodiments, the array of LEDs may be an RBGW array or an RBG array. The lamp 264 may be configured to create a light pattern to improve effects on the human circadian and melanopic system. In particular, the light patterns may generally be selected from one or more fractal-based dark-light adjacent patterns, and specifically from one or more fractal-based patterns that complement the fractal-based structure of human eye receptor layout.

In some embodiments, the lamp 264 may include an array of micromirrors to cause the light to form the pattern. In other embodiments, the lamp may include an actuator that modulates a fractal grid to create a moving, modulated output pattern. The fractal grid or other any chaotic pattern with reasonable continuous density may be disposed behind a natural fractal, such as wood grain. In still other embodiments, where the lamp that may project light onto a ceiling or surface, one or more optical properties of the light emitted by the lamp and/or one or more optic coatings on the lamp and/or the diffuser may help to modulate the light into a pattern (e.g., a dark-light adjacent pattern).

The processor 258 may be used to control the lamp 264, based at least in part on input from the camera 252. For example, the camera 252 may detect a particular area of a ceiling or wall to serve as a target for illumination; the processor 258 may control the lamp 264 to direct light only within the particular area. As other examples, the processor 258 may be used to control the lamp 264 to project light in a certain shape, in a certain color frequency or frequencies, in a certain pattern, and/or in a localized area, based on input from the camera 252. Controlling the lamp 264 in this way may help to produce light in circadian-optimized ranges.

In some embodiments, the second edge device 250 may include a speaker 266. The speaker 266 may be controlled by the processor 258 to operate as a noise machine, emitting, for example, white noise, pink noise, or the like. Additionally or alternatively, the speaker 266 may be used to output music or other soothing sounds, such as a lullaby, nature sounds, and/or the like.

The second edge device 250 may be substantially enclosed within a housing 268. The housing 268 may be formed, at least partially, from wood. In particular, at least a first portion of the housing 268 may be formed from a particular wood species veneer. The first portion of the housing 268 may act as a diffuser, modulating the light frequency of light emitted by the lamp 268 to reduce the blue content (e.g., light having a wavelength at or near 480-490 nm). In some embodiments, the housing 268 may define an aperture allowing for light emitted from the lamp 264 to exit the second edge device 250 without being modulated by the housing 268.

The housing 268 may include control point 270. In some embodiments the control point 270 may be, for example, a switch, dial, or mechanical button that a user may actuate. In other embodiments, the control point 270 may include one or more capacitive touch plates. For example, the control point 270 may comprise a touch plate formed from a natural wood material embedded with a fine meshed copper fabric to create a conductive surface just beneath the wooden surface. The control point 270 may allow for touch control of the second edge device 250. The control point 270 may be used to adjust light levels emitted by the lamp 264, turn the lamp on or off, and/or interact with the speaker 266, adjusting volume and/or turning the speaker on or off. The control point 270 preferably operates silently. The control point 270 may optionally be separated from the housing 268 by a gap sized to emit light therefrom. The gap may help to display, to a user, a position of the touch target (e.g., the control point 270). Alternatively, the control point 270 may directly abut the housing 268.

### Example 3: Platform Operation

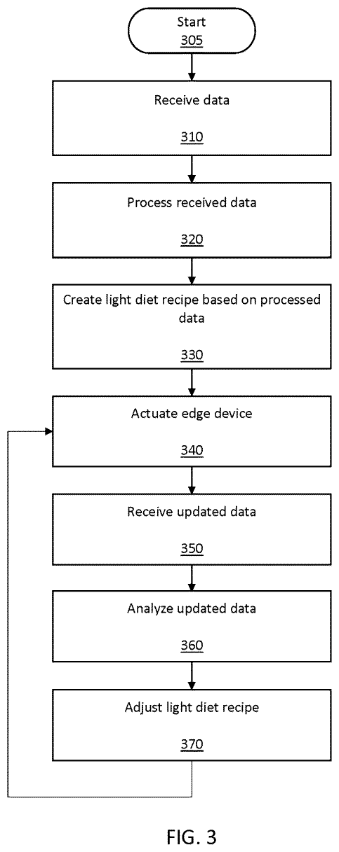

FIG. 3 is a flow chart setting forth the general stages involved in a method 300 consistent with an embodiment of the disclosure for providing therapeutic lighting, sensing, and software platform 100. Method 300 may be implemented using a computing device 400 as described in more detail below with respect to FIG. 4.

Although method 300 has been described to be performed by platform 100, it should be understood that computing device 400 may be used to perform various stages of method 300. Furthermore, in some embodiments, different operations may be performed by different networked elements in operative communication with computing device 400. For example, server 110 may be employed in the performance of some or all of the stages in method 300. Moreover, server 110 may be configured much like computing device 400. Similarly, apparatus 145 may be employed in the performance of some or all of the stages in method 300. Apparatus 145 may also be configured much like computing device 400.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 300 will be described in greater detail below.

Method 300 may begin at starting block 305 and proceed to stage 310 where the system (e.g., computing device 400) may receive data. For example, the data may include one or more user parameters and one or more user sleep goals. In embodiments, the data received by the system may include user data (e.g., identifying information, responses to a user survey, an indication of one or more user goals, an indication of one or more medical conditions that the user has, etc.) provided by the user. In embodiments, the data received may include sensor data (e.g., light levels, light spectrum information, noise levels, camera data, temperature, air pressure, etc.) from one or more sensors associated with the user. As a particular example, the one or more sensors may be included in an edge device associated with the user.

After receiving the data in stage 310, the system may proceed to stage 320, where the system may process the received data. In embodiments, processing the received data may include, for example, analyzing the received data to determine one or more external data requirements. The system may retrieve information from one or more external data sources based on the analysis.

Alternatively or additionally, processing the received data may include comparing the data received from a particular user to data received from a plurality of other users. In some embodiments, the system may determine a degree of similarity between the particular user and one or more users of the plurality of other users. In particular, the system may rely on a machine learning model to determine a similarity between the particular user and the one or more users, or the plurality of users.

From stage 320, the method 300 may advance to stage 330, where the system may create a light diet recipe associated with the particular user. The light diet recipe may be created based on, at least in part, the received data. In some embodiments, the light diet recipe may be based, at least in part, on results of the data processing, including the additional data retrieved and/or the similarity to other users, of the plurality of users.

The light diet recipe may be represented as a light algorithm for use in controlling one or more light sources associated with a user (e.g., a light source contained within an edge device associated with the user). As a particular example, the light algorithm may specify one or more of the elements in Table 2:

TABLE 2
>250 melanopic lux average during wake time till bedtime-4 h
melanopic dimming till 1 h before bedtime
30-60 minutes before bedtime light either >600 nm or metameric matching
for 480 nm ->0

In some cases, the light algorithm identifies particular light properties and particular times at which light according to the properties should be administered to the user. In some embodiments (as in the example above), the particular times may be specified with reference to a user bedtime. In other embodiments, the particular times may be specified with reference to another time (e.g., at a user wake time+8 hours) or an absolute time (e.g., at 6:00 pm).

Once computing device 400 creates the light diet recipe associated with the user in stage 330, the method 300 may continue to stage 340 where the system may cause an edge device connected to the system to operate according to the created light diet recipe. For example, the edge device may include a lamp in data communication with the system. The system may transmit commands to the edge device which cause the lamp to act according to the light algorithm.

From stage 340, method 300 may advance to stage 350, where the system may receive additional data. In some embodiments, the additional data may include updated user data, such as updated identifying information, responses to a new user survey, an indication of one or more revised user goals, an updated indication of one or more medical conditions that the user has, and/or the like. In some embodiments, the additional data may include updated sensor data from the one or more sensors associated with the user. For example, the additional sensor data may include new sensor readings related to ambient conditions in the area surrounding the user. In some embodiments, the additional data may include updated remote and correlative data. For example, the system may retrieve new data associated with the user from the one or more remote sources. As another example, the system may receive new information associated with the one or more users that have been determined to be similar to the particular user. Additionally or alternatively, the additional data may include updated sensor input from the edge device. For example, the updated sensor input may comprise one or more of a light frequency analysis and/or sound data regarding light and/or sound present in the area surrounding the edge device.

After receiving the additional data in step 350, the system may analyze the additional data in step 360. In some embodiments, analyzing the updated data may include determining at least one additional external data source is required for providing additional remote data. The system may retrieve the addition remote data from the at least one additional external data source. Additionally or alternatively, analyzing the updated data may include submitting the updated data, alone or together with the original data, to the machine learning model to determine updated degrees of similarity between the particular user and the plurality of other users.

After the system processes the additional information in stage 360, method 300 may proceed to stage 370, where the system may adjust the light diet recipe associated with the particular user based at least in part on the analyzed updated data. In particular, adjusting the light diet recipe may include adjusting a light algorithm, such that operation of the edge device is altered.

Following stage 370, the method 300 may return to stage 340, where the system may actuate the edge device based on the adjusted light diet recipe. For example, the system may transmit commands to the edge device which cause the lamp to behave according to the adjusted light diet recipe (e.g., emitting light having different wavelength(s) and/or a different light intensity).

### Example 4: Platform Architecture

Embodiments of the present disclosure provide a hardware and software platform operative as a distributed system of modules and computing elements.

The therapeutic lighting, sensing, and software platform 100 may be embodied as, for example, but not be limited to, a website, a web application, a desktop application, backend application, and a mobile application compatible with a computing device 400. The computing device 400 may comprise, but not be limited to the following:
Mobile computing device, such as, but is not limited to, a laptop, a tablet, a smartphone, a drone, a wearable, an embedded device, a handheld device, an Arduino, an industrial device, or a remotely operable recording device;
A supercomputer, an exa-scale supercomputer, a mainframe, or a quantum computer;
A minicomputer, wherein the minicomputer computing device comprises, but is not limited to, an IBM AS400/iSeries/System I, A DEC VAX/PDP, a HP3000, a Honeywell-Bull DPS, a Texas Instruments TI-990, or a Wang Laboratories VS Series;
A microcomputer, wherein the microcomputer computing device comprises, but is not limited to, a server, wherein a server may be rack mounted, a workstation, an industrial device, a raspberry pi, a desktop, or an embedded device;

Embodiments of the present disclosure may comprise a system having a central processing unit (CPU) 420, a bus 430, a memory unit 440, a power supply unit (PSU) 450, and one or more Input/Output (I/O) units. The CPU 420 coupled to the memory unit 440 and the plurality of I/O units 460 via the bus 430, all of which are powered by the PSU 450. It should be understood that, in some embodiments, each disclosed unit may actually be a plurality of such units for the purposes of redundancy, high availability, and/or performance. The combination of the presently disclosed units is configured to perform the stages any method disclosed herein.

FIG. 4 is a block diagram of a system including computing device 400. Consistent with an embodiment of the disclosure, the aforementioned CPU 420, the bus 430, the memory unit 440, a PSU 450, and the plurality of I/O units 460 may be implemented in a computing device, such as computing device 400 of FIG. 4. Any suitable combination of hardware, software, or firmware may be used to implement the aforementioned units. For example, the CPU 420, the bus 430, and the memory unit 440 may be implemented with computing device 400 or any of other computing devices 400, in combination with computing device 400. The aforementioned system, device, and components are examples and other systems, devices, and components may comprise the aforementioned CPU 420, the bus 430, the memory unit 440, consistent with embodiments of the disclosure.

At least one computing device 400 may be embodied as any of the computing elements illustrated in all of the attached figures, including the user device 105, the centralized server 110, and/or the edge device 145. A computing device 400 does not need to be electronic, nor even have a CPU 420, nor bus 430, nor memory unit 440. The definition of the computing device 400 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 400, especially if the processing is purposeful.

With reference to FIG. 4, a system consistent with an embodiment of the disclosure may include a computing device, such as computing device 400. In a basic configuration, computing device 400 may include at least one clock module 410, at least one CPU 420, at least one bus 430, and at least one memory unit 440, at least one PSU 450, and at least one I/O 460 module, wherein I/O module may be comprised of, but not limited to a non-volatile storage sub-module 461, a communication sub-module 462, a sensors sub-module 463, and a peripherals sub-module 464.

A system consistent with an embodiment of the disclosure the computing device 400 may include the clock module 410 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 420, the central component of modern computers, which relies on a clock. The only exceptions are asynchronous circuits such as asynchronous CPUs. The clock 410 can comprise a plurality of embodiments, such as, but not limited to, single-phase clock which transmits all clock signals on effectively 1 wire, two-phase clock which distributes clock signals on two wires, each with non-overlapping pulses, and four-phase clock which distributes clock signals on 4 wires.

Many computing devices 400 use a “clock multiplier” which multiplies a lower frequency external clock to the appropriate clock rate of the CPU 420. This allows the CPU 420 to operate at a much higher frequency than the rest of the computer, which affords performance gains in situations where the CPU 420 does not need to wait on an external factor (like memory 440 or input/output 460). Some embodiments of the clock 410 may include dynamic frequency change, where, the time between clock edges can vary widely from one edge to the next and back again.

A system consistent with an embodiment of the disclosure the computing device 400 may include the CPU unit 420 comprising at least one CPU Core 421. A plurality of CPU cores 421 may comprise identical CPU cores 421, such as, but not limited to, homogeneous multi-core systems. It is also possible for the plurality of CPU cores 421 to comprise different CPU cores 421, such as, but not limited to, heterogeneous multi-core systems, big.LITTLE systems and some AMD accelerated processing units (APU). The CPU unit 420 reads and executes program instructions which may be used across many application domains, for example, but not limited to, general purpose computing, embedded computing, network computing, digital signal processing (DSP), and graphics processing (GPU). The CPU unit 420 may run multiple instructions on separate CPU cores 421 at the same time. The CPU unit 420 may be integrated into at least one of a single integrated circuit die and multiple dies in a single chip package. The single integrated circuit die and multiple dies in a single chip package may contain a plurality of other aspects of the computing device 400, for example, but not limited to, the clock 410, the CPU 420, the bus 430, the memory 440, and I/O 460.

The CPU unit 420 may contain cache 422 such as, but not limited to, a level 1 cache, level 2 cache, level 3 cache or combination thereof. The aforementioned cache 422 may or may not be shared amongst a plurality of CPU cores 421. The cache 422 sharing comprises at least one of message passing and inter-core communication methods may be used for the at least one CPU Core 421 to communicate with the cache 422. The inter-core communication methods may comprise, but not limited to, bus, ring, two-dimensional mesh, and crossbar. The aforementioned CPU unit 420 may employ symmetric multiprocessing (SMP) design.

The plurality of the aforementioned CPU cores 421 may comprise soft microprocessor cores on a single field programmable gate array (FPGA), such as semiconductor intellectual property cores (IP Core). The plurality of CPU cores 421 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 421, for example, but not limited to Instruction-level parallelism (ILP) such as, but not limited to, superscalar pipelining, and Thread-level parallelism (TLP).

Consistent with the embodiments of the present disclosure, the aforementioned computing device 400 may employ a communication system that transfers data between components inside the aforementioned computing device 400, and/or the plurality of computing devices 400. The aforementioned communication system will be known to a person having ordinary skill in the art as a bus 430. The bus 430 may embody internal and/or external plurality of hardware and software components, for example, but not limited to a wire, optical fiber, communication protocols, and any physical arrangement that provides the same logical function as a parallel electrical bus. The bus 430 may comprise at least one of, but not limited to a parallel bus, wherein the parallel bus carry data words in parallel on multiple wires, and a serial bus, wherein the serial bus carry data in bit-serial form. The bus 430 may embody a plurality of topologies, for example, but not limited to, a multidrop/electrical parallel topology, a daisy chain topology, and a connected by switched hubs, such as USB bus. The bus 430 may comprise a plurality of embodiments, for example, but not limited to:
Internal data bus (data bus) 431/Memory bus.
Control bus 432.
Address bus 433.
System Management Bus (SMBus).
Front-Side-Bus (FSB).
External Bus Interface (EBI).
Local bus.
Expansion bus.
Lightning bus.
Controller Area Network (CAN bus).
Camera Link.
ExpressCard.
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS).
HyperTransport.
InfiniBand.
RapidIO.
Mobile Industry Processor Interface (MIPI).
Coherent Processor Interface (CAPI).
Plug-n-play.
1-Wire.
Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v2], PCI Express External Cabling [ePCIe], and PCI Express OCuLink [Optical Copper{Cu}Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/104 bus (e.g., PC/104-Plus, PCI/104-Express, PCI/104, and PCI-104), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1394 Interface/Firewire, Thunderbolt, and eXtensible Host Controller Interface (xHCI).

Consistent with the embodiments of the present disclosure, the aforementioned computing device 400 may employ hardware integrated circuits that store information for immediate use in the computing device 400, know to the person having ordinary skill in the art as primary storage or memory 440. The memory 440 operates at high speed, distinguishing it from the non-volatile storage sub-module 461, which may be referred to as secondary or tertiary storage, which provides slow-to-access information but offers higher capacities at lower cost. The contents contained in memory 440, may be transferred to secondary storage via techniques such as, but not limited to, virtual memory and swap. The memory 440 may be associated with addressable semiconductor memory, such as integrated circuits consisting of silicon-based transistors, used for example as primary storage but also other purposes in the computing device 400. The memory 440 may comprise a plurality of embodiments, such as, but not limited to volatile memory, non-volatile memory, and semi-volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 441, Static Random-Access Memory (SRAM) 442, CPU Cache memory 425, Advanced Random-Access Memory (A-RAM), and other types of primary storage such as Random-Access Memory (RAM).
Non-volatile memory which can retain stored information even after power is removed, for example, but not limited to, Read-Only Memory (ROM) 443, Programmable ROM (PROM) 444, Erasable PROM (EPROM) 445, Electrically Erasable PROM (EEPROM) 446 (e.g., flash memory and Electrically Alterable PROM [EAPROM]), Mask ROM (MROM), One Time Programable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).

Consistent with the embodiments of the present disclosure, the aforementioned computing device 400 may employ the communication system between an information processing system, such as the computing device 400, and the outside world, for example, but not limited to, human, environment, and another computing device 400. The aforementioned communication system will be known to a person having ordinary skill in the art as I/O 460. The I/O module 460 regulates a plurality of inputs and outputs with regard to the computing device 400, wherein the inputs are a plurality of signals and data received by the computing device 400, and the outputs are the plurality of signals and data sent from the computing device 400. The I/O module 460 interfaces a plurality of hardware, such as, but not limited to, non-volatile storage 461, communication devices 462, sensors 463, and peripherals 464. The plurality of hardware is used by the at least one of, but not limited to, human, environment, and another computing device 400 to communicate with the present computing device 400. The I/O module 460 may comprise a plurality of forms, for example, but not limited to channel I/O, port mapped I/O, asynchronous I/O, and Direct Memory Access (DMA).

Consistent with the embodiments of the present disclosure, the aforementioned computing device 400 may employ the non-volatile storage sub-module 461, which may be referred to by a person having ordinary skill in the art as one of secondary storage, external memory, tertiary storage, off-line storage, and auxiliary storage. The non-volatile storage sub-module 461 may not be accessed directly by the CPU 420 without using intermediate area in the memory 440. The non-volatile storage sub-module 461 does not lose data when power is removed and may be two orders of magnitude less costly than storage used in memory module, at the expense of speed and latency. The non-volatile storage sub-module 461 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (461) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory.
Holographic data storage such as Holographic Versatile Disk (HVD).
Molecular Memory.

### Example 5: Deoxyribonucleic Acid (DNA) Digital Data Storage

Consistent with the embodiments of the present disclosure, the aforementioned computing device 400 may employ the communication sub-module 462 as a subset of the I/O 460, which may be referred to by a person having ordinary skill in the art as at least one of, but not limited to, computer network, data network, and network. The network allows computing devices 400 to exchange data using connections, which may be known to a person having ordinary skill in the art as data links, between network nodes. The nodes comprise network computer devices 400 that originate, route, and terminate data. The nodes are identified by network addresses and can include a plurality of hosts consistent with the embodiments of a computing device 400. The aforementioned embodiments include, but not limited to personal computers, phones, servers, drones, and networking devices such as, but not limited to, hubs, switches, routers, modems, and firewalls.

Two nodes can be said are networked together, when one computing device 400 is able to exchange information with the other computing device 400, whether or not they have a direct connection with each other. The communication sub-module 462 supports a plurality of applications and services, such as, but not limited to World Wide Web (WWW), digital video and audio, shared use of application and storage computing devices 400, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 4 [IPv4], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 462 may comprise a plurality of size, topology, traffic control mechanism and organizational intent. The communication sub-module 462 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G,4G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line communications.

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 400 may employ the sensors sub-module 463 as a subset of the I/O 460. The sensors sub-module 463 comprises at least one of the devices, modules, and subsystems whose purpose is to detect events or changes in its environment and send the information to the computing device 400. Sensors are sensitive to the measured property, are not sensitive to any property not measured, but may be encountered in its application, and do not significantly influence the measured property. The sensors sub-module 463 may comprise a plurality of digital devices and analog devices, wherein if an analog device is used, an Analog to Digital (A-to-D) converter must be employed to interface the said device with the computing device 400. The sensors may be subject to a plurality of deviations that limit sensor accuracy. The sensors sub-module 463 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).
Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 400 may employ the peripherals sub-module 462 as a subset of the I/O 460. The peripheral sub-module 464 comprises ancillary devices uses to put information into and get information out of the computing device 400. There are 3 categories of devices comprising the peripheral sub-module 464, which exist based on their relationship with the computing device 400, input devices, output devices, and input/output devices. Input devices send at least one of data and instructions to the computing device 400. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.

The number of degrees of freedom involved, such as, but not limited to, two-dimensional mice vs three-dimensional mice used for Computer-Aided Design (CAD) applications.

Output devices provide output from the computing device 400. Output devices convert electronically generated information into a form that can be presented to humans. Input/output devices perform that perform both input and output functions. It should be understood by a person having ordinary skill in the art that the ensuing are non-limiting embodiments of the aforementioned peripheral sub-module 464:

### Input Devices

Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).

High degree of freedom devices, that require up to six degrees of freedom such as, but not limited to, camera gimbals, Cave Automatic Virtual Environment (CAVE), and virtual reality systems.

Video Input devices are used to digitize images or video from the outside world into the computing device 400. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.

Audio input devices are used to capture sound. In some cases, an audio output device can be used as an input device, in order to capture produced sound. Audio input devices allow a user to send audio signals to the computing device 400 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.

Data AcQuisition (DAQ) devices convert at least one of analog signals and physical parameters to digital values for processing by the computing device 400. Examples of DAQ devices may include, but not limited to, Analog to Digital Converter (ADC), data logger, signal conditioning circuitry, multiplexer, and Time to Digital Converter (TDC).

Output Devices may further comprise, but not be limited to:

Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).

Printers, such as, but not limited to, inkjet printers, laser printers, 3D printers, solid ink printers and plotters.

Audio and Video (AV) devices, such as, but not limited to, speakers, headphones, amplifiers and lights, which include lamps, strobes, DJ lighting, stage lighting, architectural lighting, special effect lighting, and lasers.

Other devices such as Digital to Analog Converter (DAC).

Input/Output Devices may further comprise, but not be limited to, touchscreens, networking device (e.g., devices disclosed in network 462 sub-module), data storage device (non-volatile storage 461), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method comprising:
receiving, at a computing device, user data specifying user parameters and user sleep, health and wellness goals;
creating, based on the user data, a light algorithm associated with the user;
causing an edge device connected to the centralized server to operate according to the created light algorithm;
receiving, at the computing device, sensor input from the edge device; and
adjusting operation of the edge device based on the received sensor input.

2. The method of claim 1, further comprising:
creating, based on the user data, a user schedule; and
transmitting the user schedule to a user device.

3. The method of claim 1, wherein the user data comprises one or more of the following:
user parameter data specifying one or more parameters associated with the user; and
user goal data specifying one or more user goals.

4. The method of claim 1, wherein the sensor input comprises one or more of the following:
image data from a camera connected to the edge device; and
audio data from a microphone connected to the edge device.

5. The method of claim 1, wherein the sensor data comprises data indicating that a user is awake.

6. One or more non-transitory computer readable media comprising instructions which, when executed by one or more hardware processors, causes performance of operations comprising:
receiving, at a computing device, user data specifying user parameters and user sleep, health and wellness goals;
creating, based on the user data, a light algorithm associated with the user;
causing an edge device connected to the centralized server to operate according to the created light algorithm;
receiving, at the computing device, sensor input from the edge device; and
adjusting operation of the edge device based on the received sensor input.

7. The non-transitory computer readable media of claim 6, further comprising:
creating, based on the user data, a user schedule; and
transmitting the user schedule to a user device.

8. The non-transitory computer readable media of claim 6, wherein the user data comprises one or more of the following:
user parameter data specifying one or more parameters associated with the user; and
user goal data specifying one or more user goals.

9. The non-transitory computer readable media of claim 6, wherein the sensor input comprises one or more of the following:
image data from a camera connected to the edge device; and
audio data from a microphone connected to the edge device.

10. The non-transitory computer readable media of claim 6, wherein the sensor data comprises data indicating that a user is awake.

11. A system comprising:
at least one device including a hardware processor;
the system being configured to perform operations comprising:
receiving, at a computing device, user data specifying user parameters and user sleep, health and wellness goals;
creating, based on the user data, a light algorithm associated with the user;
causing an edge device connected to the centralized server to operate according to the created light algorithm;
receiving, at the computing device, sensor input from the edge device; and
adjusting operation of the edge device based on the received sensor input.

12. The system of claim 11, the operations further comprising:
creating, based on the user data, a user schedule; and
transmitting the user schedule to a user device.

13. The system of claim 11, wherein the user data comprises one or more of the following:
user parameter data specifying one or more parameters associated with the user; and
user goal data specifying one or more user goals.

14. The system of claim 11, wherein the sensor input comprises one or more of the following:
image data from a camera connected to the edge device; and
audio data from a microphone connected to the edge device.

15. The system of claim 11, wherein the sensor data comprises data indicating that a user is awake.

16. A therapeutic lighting, sensing, and software system comprising:
a camera;
a microphone;
a light source;
a computing device in signal communication with the camera and the microphone, the computing device being configured to control at least an intensity of light emitted by the light source based on input from one or more of the camera or the microphone; and
a light filtering enclosure surrounding at least the light source, the light filtering enclosure configured to restrict light emissions that would trigger a melanopic reaction in humans; and
a machine learning computing device applying artificial intelligence (AI) including machine learning to analyze at least one of user data and device data in order to gain usage and effectiveness insights and create predictions relating to future usage and effectiveness.

17. The system of claim 16, wherein the light filtering enclosure is configured to prevent light having a wavelength of about 480-490 nm from passing through the enclosure.

18. The system of claim 16, wherein the light filtering enclosure is configured to permit light having a wavelength of about 620-650 nm to pass through the enclosure.

19. A therapeutic lighting, sensing, and software system comprising:
a camera;
a microphone;
a light source;
a computing device in signal communication with the camera and the microphone, the computing device being configured to control at least an intensity of light emitted by the light source based on input from one or more of the camera or the microphone; and
a light filtering enclosure surrounding at least the light source, the light filtering enclosure configured to restrict light emissions that would trigger a melanopic reaction in humans; and
a machine learning computing device applying artificial intelligence (AI) including machine learning to analyze at least one of user data and device data in order to gain usage and effectiveness insights and create predictions relating to future usage and effectiveness.

20. The system of claim 19, wherein the light filtering enclosure is configured to prevent light having a wavelength of about 480-490 nm from passing through the enclosure.

21. The system of claim 19, wherein the light filtering enclosure is configured to permit light having a wavelength of about 620-650 nm or more to pass through the enclosure.

22. One or more non-transitory computer readable media comprising instructions which, when executed by one or more hardware processors, causes performance of operations comprising:
receiving user data associated with a user;
receiving ambient data from one or more of a camera or a microphone, wherein the received ambient data is associated with the user;
processing the user data and the ambient data to create a light diet; and
controlling a light source based on the received light diet.

23. The non-transitory computer readable media of claim 22, wherein the light diet specifies one or more of:
an intensity of light to be emitted by the light source, or
a range of wavelength of light to be emitted by the light source.

24. The non-transitory computer readable media of claim 22, wherein processing the user data and the ambient data comprises supplying at least a portion of one or more of the user data and the ambient data as inputs to a machine learning model, and wherein the machine learning model provides the light diet as an output.
